(19) United States
(12) Patent Application Publication
(10) Pub. No.: US 2024/0399017 A1
(43) Pub. Date: Dec. 5, 2024

(54) AIR PURIFIER

(71) Applicant: Airea, LLC, Chicago, IL (US)

(72) Inventors: Scott H. Wilson, Chicago, IL (US); Gary H. Paulsen, Chicago, IL (US); D. Matthew Puhalla, Glen Ellyn, IL (US)

(21) Appl. No.: 18/674,717

(22) Filed: May 24, 2024

Related U.S. Application Data
(63) Continuation-in-part of application No. 18/328,559, filed on Jun. 2, 2023.

(51) Int. Cl. A61L 9/20; B01D 53/00; B01D 53/88

(52) U.S. Cl. CPC A61L 9/205; B01D 53/007; B01D 53/885; A61L 2209/11; A61L 2209/12; B01D 2255/802; B01D 2257/91; B01D 2259/804

(57) ABSTRACT

An air purification unit has an air inlet, an air outlet separated a distance from the air inlet, and an ultraviolet light source positioned along a length of the distance. The air purification unit also has a removable ionization cell comprising an inner surface coated with a catalyst and an outer surface, wherein the removable ionization cell has an unfolded position where the outer surface is substantially flat, and a folded position in which the inner surface is configured to substantially surround the ultraviolet light source.

### CROSS-REFERENCES TO RELATED APPLICATIONS

This patent application is a continuation-in-part of application Ser. No. 18/328,559, filed Jun. 2, 2023. application Ser. No. 18/328,559 is incorporated into the present disclosure by this reference.

### TECHNICAL FIELD

This invention relates generally to air purification systems, and more specifically, to air purification systems and devices that use ultraviolet light to induce oxidation to destroy germs, microbes, and other small airborne pathogens.

### BACKGROUND

Air purifiers are a known tool for not only removing odors but also for helping to sterilize and cleanse the air in a room. They have become more prevalent and in demand after the recent COVID-19 pandemic, and as there are more concerns over airborne pathogens. The simplest and traditional form of an air purifier is simply a fan and a filter captured within a housing. The fan draws air in, passes it through a filter to remove dust and other larger particles, and circulates the cleaner air back out into the room. Essentially all residential and commercial HVAC systems include serviceable/replaceable air filters that perform this air cleaning function. However, filters only catch larger particles and do nothing to halt the spread of smaller microbes, viruses, and bacteria.

To cleanse the air of these smaller particles, a process of oxidation can be performed. Oxidation is a chemical reaction that combines a substance with oxygen to change its properties. This chemical reaction can be used to kill or render inert bacteria, mold, viruses, and other small harmful products that may exist in residential or commercial room air. While oxidation occurs naturally in the presence of sunlight, air, and water, it can be greatly accelerated in the confines of an air purification device using a process called photocatalysis. Photocatalysis is a rapid, forced oxidation process where broad spectrum ultraviolet light is cast upon a hydrophilic surface coated with a catalyzing agent that absorbs moisture from the surrounding air to produce hydrogen peroxide ions. These ions quickly break down into water and air, and in the process reduce air pollutants and neutralize viruses. An example of this process is disclosed in U.S. Pat. No. 7,988,923 to Fink, which is incorporated herein by reference in its entirety. Though other agents can be used, Fink discloses use of a “quad metallic” catalyzing agent on the hydrophilic surface that includes titanium oxide (TiO2), copper, sulfur and rhodium combined with a hydrating agent such as silica gel.

Photocatalysis has been harnessed and used in large, industrial air purification systems, and has proven effective at reducing or eliminating deadly viruses such as SARS-COV-1 (protein jacketed), H1N1 (Swine Flu), H5N1 (Bird flu), as well as to quickly and effectively remove odors in the air. However, it has not been harnessed in a suitable residential air purification device. This is, at least in part, because of the cost and maintenance requirements of traditional systems, as well as the hazards from the ultraviolet light source itself, which is very bright and can be harmful to the human eye and skin. What is needed is a practical design that brings this process of photocatalysis within reach of the common consumer.

Configurations of the disclosed technology address shortcomings in the prior art.

### SUMMARY OF THE DISCLOSURE

The present invention provides for air purification systems and devices that provide photocatalysis in a consumer-friendly form that is capable of both protecting users from harmful UV light exposure and cleansing a large amount of room air with a compact device that is easily serviced.

In some embodiments, the air purification system includes air purification unit with an ultraviolet light source and an ionization cell having an inner surface coated with a catalyst. The inner surface forms a series of ridges and valleys where each valley has at least one foldable seam that allows the ionization cell to fold at least partially around the ultraviolet light source. A fan assembly is positioned to create an airflow along the inner surface of the ionization cell. The ionization cell may be fitted within a rigid sleeve that fits into a housing to position it around the ultraviolet light source. The air purification unit may include a base structure for housing the fan and containing a motor and electric source for powering the fan. The base structure may be configured to allow air to flow in through the fan and through the ionization cell. An opaque removable cover may be placed over the ionization cell to block exposure to ultraviolet light.

In other embodiments, the air purification system includes an air purification unit having an air inlet, an air outlet separated a distance from the air inlet, and an ultraviolet light source positioned along a length of the distance. The unit has a removable ionization cell with an inner surface coated with a catalyst and an outer surface, where the removable ionization cell has an unfolded position in which the outer surface is substantially flat and a folded position in which the inner surface is configured to substantially surround the ultraviolet light source. The unit may include a fan to move air along the ionization cell and may include a switch to control the flow of electricity to the ultraviolet light source, wherein the switch only allows the flow of electricity to the ultraviolet light source when a substantially opaque removable cover is fit over the ultraviolet light source.

In still other embodiments, the air purification system includes an air purification unit with a base structure, a removable outer cover configured to fit to the base structure, an ultraviolet light source connected to the base structure, and an ionization cell positioned substantially surrounding the ultraviolet light source, the ionization cell having an inner surface facing the ultraviolet light source and an outer surface facing away from the ultraviolet light source, wherein the inner surface is coated with a catalyst and forms a series of ridges and valleys such that the ridges extend toward the ultraviolet light source and the valleys extend away from the ultraviolet light source. The ionization cell may have at least one foldable seem along at least one valley such that, when the ionization cell is removed from the air purification unit, it can be unfolded along the at least one foldable seam.

As will be understood and appreciated by those of skill in the art from a review of the full written description below, variations may be made to the component configurations described above in some embodiments, and additional components may be used in some particular embodiments. For example, some embodiments may be incorporated into a mobile air purifier unit designed to sit on a table or in a fixed air purifier unit designed to be mounted to a wall. Still other embodiments and/or features are identified in the disclosure and claims below, in combination with the associated figures.

### BRIEF DESCRIPTION OF THE DRAWINGS

The present invention can be better understood with reference to the following drawings. The components in the drawings are not necessarily to scale, emphasis instead being placed upon clearly illustrating the principles of the present invention. Where dimensions are provided, they are used for reference and understanding, and are not limiting unless the feature in question expressly claimed to be of a particular dimension. In the drawings, like reference numerals designate corresponding parts throughout the several views.

FIG. 1 is a perspective view of an air purification unit in accordance with the present invention.

FIG. 2 is an exploded view of the air purification unit of FIG. 1, showing various internal components.

FIG. 3 is a perspective view of certain components of the air purification unit of FIG. 1, exploded to show how the components come together.

FIG. 4 is a perspective view of an opaque cover component of the air purification unit of FIG. 1 taken from a first side angle.

FIG. 5 is a perspective view of the opaque cover component shown in FIG. 4 but taken from a steeper side angle to show the interior thereof.

FIG. 6 is an exploded view of a core assembly of an air purification device, in accordance with an embodiment of the present invention.

FIG. 7 is an isometric view of an ionization cell in an unfolded condition, in accordance with an embodiment of the present invention.

FIG. 8 is an isometric view of the core assembly of FIG. 6 in an assembled condition.

FIG. 9 is an isometric view of a core assembly in an assembled condition in accordance with an alternative embodiment.

FIG. 9A is a top view of the core assembly of FIG. 9.

FIG. 10 is a perspective view of a filter assembly for an air purification device in accordance with certain embodiments of the present invention.

FIG. 11 is an exploded view of a base assembly of an air purification device in accordance with certain embodiments of the present invention.

FIG. 12 is a bottom view of the base assembly of FIG. 11 in a fully assembled condition.

FIG. 13 is a perspective view of the base assembly of FIG. 12.

FIG. 14 is a section view of the air purification unit of FIG. 1, taken down the center of the unit along section line A.

FIG. 15 is a front side perspective view of an air purification unit in accordance with another embodiment of the present invention.

FIG. 16 is a rear side perspective view of the air purification unit shown in FIG. 15.

FIG. 17 is a front side perspective view of the air purification unit of FIG. 15 with the front cover removed and the internal components in an operating condition.

FIG. 18 is a front side perspective view of the air purification unit of FIG. 15 with the front cover removed and the internal components in a service position.

FIG. 19 is an exploded view of the air purification unit of FIG. 18 showing certain components extended out from the unit.

FIG. 20 is an exploded view of an air purification unit implementing a core assembly, according to an additional configuration.

FIG. 21 is a perspective view of the assembled air purification unit and core assembly of FIG. 20.

FIG. 22 is a perspective view of an ionization cell for use with the core assembly of FIGS. 20-21, shown in an unfolded position.

FIG. 23 is a top view of the ionization cell of FIG. 22, shown in an unfolded position.

FIG. 24 is a top view of the ionization cell of FIG. 22, shown in a folded position.

FIG. 25 is a top view of the core assembly of FIGS. 20-21.

FIG. 26 is a cross-sectional view of the core assembly of FIG. 25.

### DETAILED DESCRIPTION

The description that follows describes, illustrates, and exemplifies one or more particular embodiments of the present invention in accordance with its principles. This description is not provided to limit the invention to the embodiments described herein, but rather to explain and teach the principles of the invention in such a way to enable one of ordinary skill in the art to understand these principles and, with that understanding, be able to apply them to practice not only the embodiments described herein, but also other embodiments that may come to mind in accordance with these principles. The scope of the present invention is intended to cover all such embodiments that may fall within the scope of the appended claims, either literally or under the doctrine of equivalents.

The air purification units described herein may take on various physical forms and still fall within the scope of the claims set forth herein. For purposes of example, a subset of possible formats is disclosed. The units may be portable or fixed in position, depending on the physical form and other characteristics such as disclosed herein. One of ordinary skill in the art of mechanical systems will understand that the size of the exemplary embodiments disclosed, and of the air purification units claimed, are scalable. That is, a larger unit would be able to cleanse a larger volume of air in a given period of time than a smaller unit. Moreover, larger or smaller units could be built based on the teachings herein simply by scaling the components of the unit, such as the ultraviolet light source, the fan, and the core assembly, as discussed further below. Testing shows that a unit having a core assembly 120 mm in length can sufficiently cleanse the air in an 800 square foot room with a standard 8-foot ceiling.

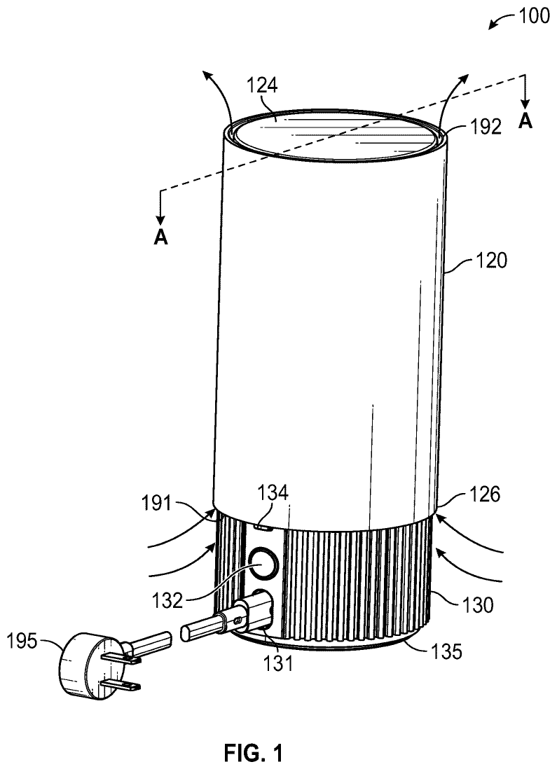

FIG. 1 shows a perspective view of an air purification unit 100 in accordance with particular embodiments of the present invention. The air purification unit 100, also referred to herein as the “mobile unit,” features a base structure 130 and an outer cover 120 that come together to form an interior housing. As will be discussed, it is helpful for both of these components to be essentially opaque so as to prevent external exposure to the ultraviolet light source contained within. Arrows show that, in this particular embodiment, room air is pulled up under the lower perimeter 126 of the outer cover 120 and eventually exits out air outlets 192 formed in a top surface of the cover 120. As will be explained, it is between this lower perimeter 126 and the air outlets 192 that the air is exposed to and cleansed by hydrogen peroxide ions.

Air purification unit 100 is designed to be a portable embodiment that can be placed on a table (typically smaller units) or on the floor (typically larger units). It requires a power source, which in this case is supplied by an electrical cable 195 that plugs into a standard wall outlet. The other end of the cable 195 plugs into a power receptacle 131 near the bottom of the base structure 130. The air purification unit sits on a bottom surface 135 of the base structure 130. In some embodiments, the base structure 130 may alternatively or additionally house a battery pack that may store electricity such that the air purification unit 100 may operate temporarily in the absence of external power. It will be understood that the unit 100 could alternatively be powered via an adapter via a USB port, a cigarette lighter of a vehicle, or other common power source.

Air purification unit 100 is further equipped with a power button 132 for turning the unit “on” by supplying electricity to the internal components. Alternatively or additionally, the unit 100 could contain an air quality sensor that determines when to automatically turn the unit on and off based on a variety of metrics such as air density, temperature, or the detection of certain types of dust or other particles in the air. Alternatively or additionally, the unit 100 could be equipped with a timer that causes the unit to operate during certain times of the day and for certain periods. In some instances, the operational timing could be adjusted manually by a user either via on-unit controls (not shown) or via a computer-based application that presents a control interface, such as on a user's mobile computer device (e.g., an i-phone from Apple). In such a case, the mobile computer device would convey instructions provided by the user to the air purification device via a wireless connection such as Bluetooth or the like, as is well known in the art of smart home appliances. In such cases, the air purification unit 100 would be further equipped with a receiver or transceiver for receiving and/or sending communications, such as via Bluetooth, from and/or to the mobile device.

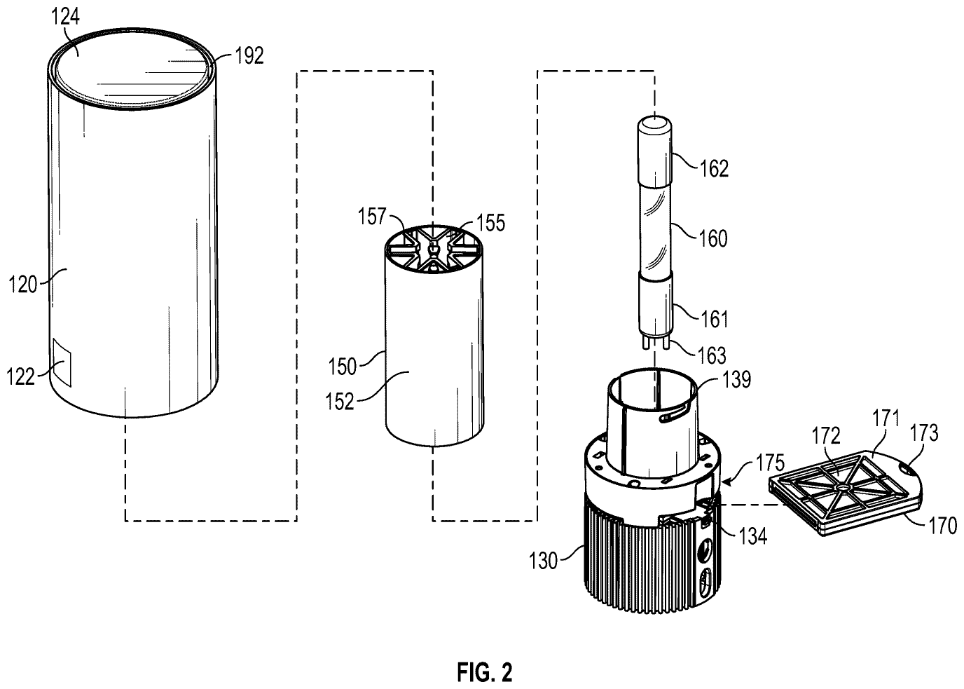

Another feature of air purification unit 100 visible in FIG. 1 is the safety catch 134. Safety catch 134 functions as a toggle switch that controls the supply of electricity to an interior ultraviolet light source, as further shown below. When the outer cover 120 is installed over the base structure 130, the safety catch 134 is depressed, which allows power to reach the light source 160 (see FIG. 2) when the air purification unit 100 is powered on. When the outer cover 120 is removed, such as or servicing of the ionization cell 157 or to change the bulb 160, the safety catch 134 rises to a free position that causes electricity to the bulb to be cut off. This protects users and people nearby from being accidentally exposed to ultraviolet light when the outer cover 120 is removed and allows inspection of the unit 100 while still powered on.

FIG. 2 shows an exploded view of the air purification unit 100 so as to expose various internal components. Outer cover 120 has been lifted away and is off to the right. As rotated in this view, label 122 is in view, which can simply be a brand label of the device. Base structure 130 has maintained position and is on the right, with bulb 160 extended above it. Because the outer cover 120 has been removed from the base structure 130, the safety catch 134 is in its free (raised) position, which again cuts power to the bulb 160. Bulb 160 serves as the ultraviolet light source in the illustrated embodiment. It will be understood that various ultraviolet light sources capable of producing light waves in the 200-300 nm wavelength range can be used. For example, bulb 160 is modeled after a four pin 250 UV light bulb designed for germicidal water treatment and made by Ultra Dynamics. As shown, bulb 160 has a bottom cap 161 and a top cap 162 that are generally opaque, and a center portion in between that is translucent (typically glass or clear plastic), such that light only emanates from the bulb 160 between the caps 161, 162.

The bulb 160 fits inside core assembly 150, which is further exploded above and away from the bulb in FIG. 2. As discussed further below, the core assembly contains a catalyst surface 155 on one side of an ionization cell 157 that wraps around the exposed center portion of bulb 160 when assembled. In operation, both the core assembly 150 and the bulb fit into core assembly housing 139 of the base structure, and the bulb 160 plugs into light receptacle 146.

Off to the right is a filter assembly 170 that operates like a filter in a standard air duct. When installed, the filter assembly 170 fits into the filter tray housing 175 carved out of the base structure 130. The filter assembly 170 can be positioned on either side of a fan (as shown below) and used to cleanse the air of larger inert particles, leaving only smaller viruses, microbes, odor-causing agents, etc., to pass through the core assembly 150. In some embodiments the air filter (and or the fan) may be located on the other side of the core assembly 150

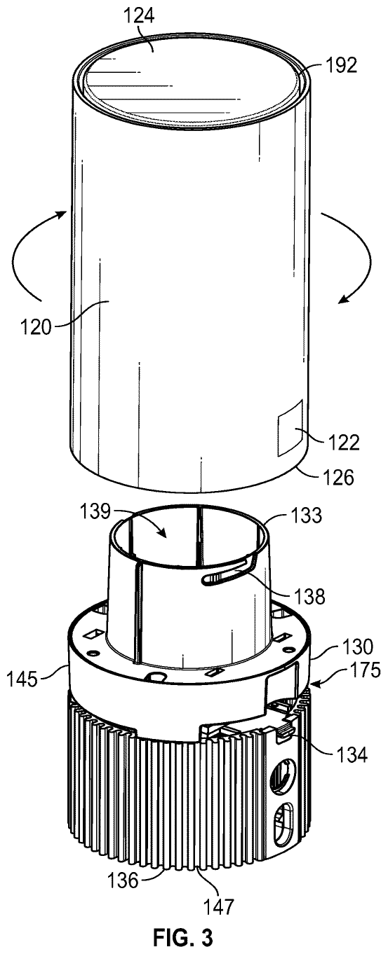

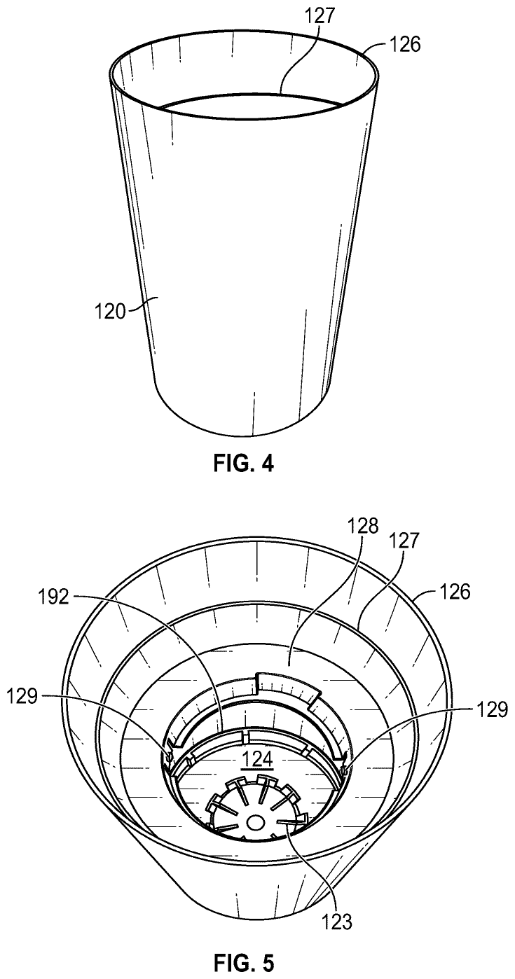

FIG. 3 is perspective view of the base structure 130 with the outer cover 120 removed extended above it. As shown, the base structure 130 features an upward extension that forms the core assembly housing 139. Cut into the outer surface of the upward extension is a rotational groove 138. As shown best in FIG. 4, these grooves correspond with protrusions 129 on the inside of the outer cover 120 such that the protrusions 129 fit into the grooves 138 to draw the outer cover 120 down over the base structure 130 into an assembled condition as it twists clockwise (see arrows). The lower perimeter 126 of the outer cover 120 will then press the safety catch 134 down so that the ultraviolet light source (bulb 160) can be engaged.

The lower perimeter 126 is large enough in diameter to fit down over the fan housing 145 of the base structure 130 and comes to rest just over the set of undulating ridges formed into the outer wall of the base structure 130. The ridges form a series of undulating base structure extensions 136 and protrusions 147 around the perimeter of base structure 130, which provide channels (along the protrusions) for the passage of room air up and under the lower perimeter 126 of the outer cover 10. The lower perimeter 126 has an inner diameter that approximately matches the distance from one base structure extension 136 laterally across to an opposing extension 136 on the far side of the base structure 130.

FIGS. 4 and 5 are perspective views of the removed outer cover 120, showing it at different angles to reveal its inside features. The features are formed to cooperate with corresponding features on the base structure 130 and core assembly 150. For example, lower step 127 seats against the upper surface of fan housing 145 when the outer cover 120 is twisted into place over the base structure 130. This helps close off air from escaping around the core assembly and generally helps create suction to pull it through the fan assembly 140 and up into the core assembly 150. Meanwhile, upper step 128, which is much wider than lower step 127, closes off around the outer perimeter of the extension that forms the core assembly housing 139 of the base structure 130. Again, this provides to close off air escape passageways and also provides for structural rigidity of the unit 100 when assembled.

In FIG. 5, additional features formed on the inside of the top surface 124 can be seen. These features include a series of cell spacers 123 that correspond to the shape of the ionization cell 157 (discussed below) when it is in its folded configuration. This helps securely hold the ionization cell in place within its sleeve 152. Again, a series of holes or slots form the air outlet 192 around the top perimeter of the outer cover 120.

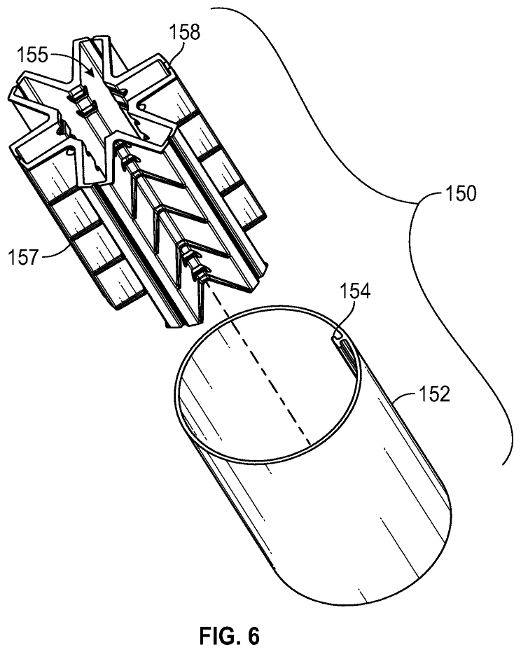

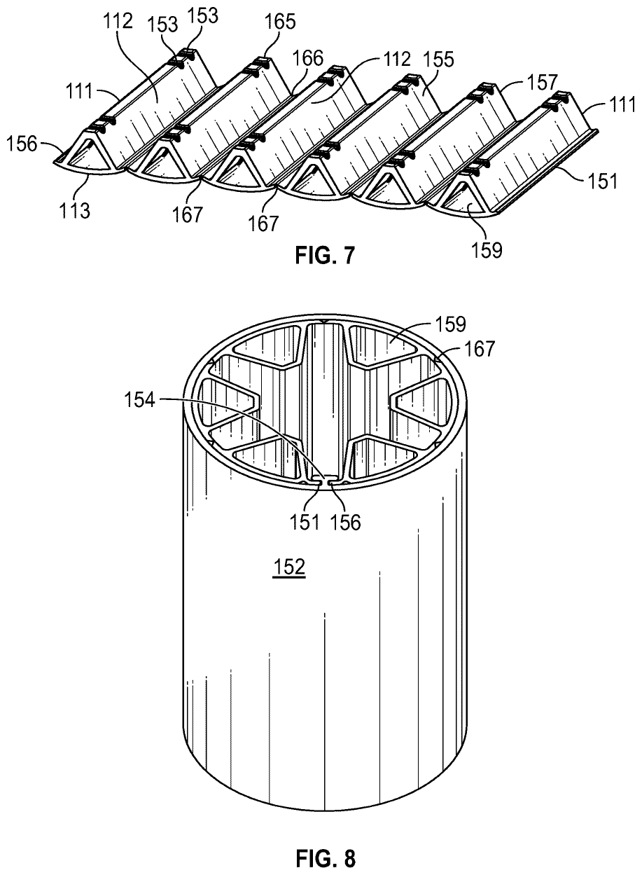

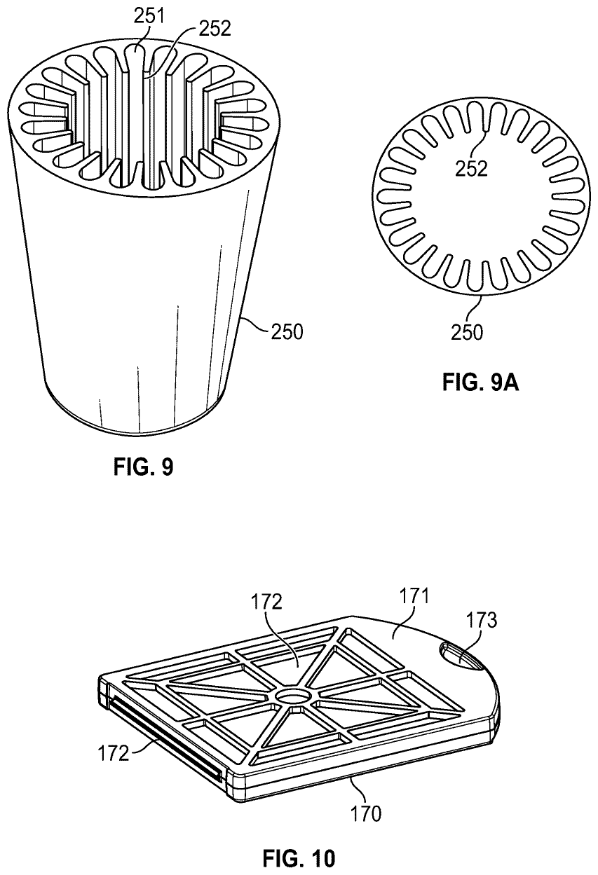

FIG. 6 is an exploded isolation view of the core assembly, showing its two primary components the sleeve 152 and the ionization cell 157. The sleeve 152 is a rigid tube in which the ionization cell 157 is fit when in its folded configuration. While the cell spacers 123 on the inside surface of the outer shell 120 help hold the ionization cell 157 in place, the sleeve 152 features a rib 154 to help prevent rotation of the cell 157. As more clearly understood in view of FIGS. 7 and 8, the ionization cell 157 has first and second edges 151, 156 that come in close proximity when the ionization cell 157 is in its folded configuration, such that these edges 151, 156 fit underneath opposing sides of the rib 154 as the cell 157 is slid into the sleeve 152. FIG. 8 shows the ionization cell 157 assembled within the sleeve 152, and shows how the first and second edges 151, 156 fit along the rib 154.

The ionization cell 157 has a series of flexible seams 167, also called “living hinges,” that allow the cell 157 to transition between a folded configuration or position and an unfolded configuration or position. The seams may be made of the same material as the rest of the ionization cell 157, but have a necked-down, thinner profile that allows for a high level of flexibility. For example, while scalable, the regular thickness of the ionization surfaces is preferably between 1 and 2 millimeters, while the thickness of the “living hinge” seam 167 is preferably between 0.3 to 0.5 millimeters.

FIG. 7 shows the ionization cell 157 in its unfolded position. The ionization cell may be made of a semi-flexible material such as polycarbonate, polyamide or polypropylene. Other plastics, paper pulp or other natural and compostable materials could also be used. Material selection will ideally allow an increased level of flexibility at the seam 167 and a more firm shape for the remainder of the cell 157. For example, the cell 157 should hold its shape as it is inserted into or extracted from the sleeve 152. While manipulation of the seams 167 will typically be infrequent, the material and thickness should allow for hundreds of folds and unfolds without tearing. Also, the material must be able to withstand heat and radiation from the ultraviolet light source over long periods of time.

The ionization cell 157 is coated, at least on its upper surface (as shown in FIG. 7), with a catalyst designed to interact with high intensity ultraviolet light to produce hydrogen peroxide ions and other oxidizing agents. An example of such a catalyst is the “quad metallic” coating described in Fink (U.S. Pat. No. 7,988,923), which consists of titanium oxide, copper, silver and rhodium. The foldable/unfolded nature of the ionization cell 157 makes it very easy to consistently apply this coating during the manufacturing process and avoid application on surfaces facing away from the UV light source where it has little or no value and is largely wasted. For example, the coating may be applied to ionization cell 157 with a spray process as opposed to dipping, which would coat the entire exterior surface.

In between each foldable seam 167 is a cell component 111 that features an upward-extending (in the unfolded position) or inward-extending (in the folded position) central ridge 165 and opposing slopes 112 falling away to either side of the ridge 165 into a trough 166 formed between each cell component 111. Each cell component 111 is hollow underneath the ridge 165 and has a backing element 113 that forms an arc connecting the two opposing slopes 112 and extending slightly beyond the slope ends to a foldable seam 167 on either side. When in the folded configuration, these backing elements 113 approximately form a cylinder with a diameter configured to fit inside the sleeve 152. Also in this configuration, the ridges 165 combine to form a relatively tight pocket for the UV bulb 160 to drop into (See FIG. 2). When installed, it is preferably to have only one to three millimeters of separation between the bulb 160 and the ridges 165. This design achieves the goal of presenting the catalyst very proximate to the UV light source, i.e., on the top of the ridges 165, while also maximizing catalyst-coated surface area exposed to the light, i.e., with the slopes 112 and troughs 166.

The hollow sections 159 formed by each cell component 111 allow for additional passage of air through the core assembly. Air passing through these sections 159 does not come into as intense contact with the oxidizing agents created by the UV light reacting with the surface coating but does still get sufficiently ionized and allows for greater output and quieter operation. This is assisted by holes 153 positioned along the ridges 165 of the cell components 111, which allow air to pass back and forth on either side of the ridges and also allows hydrogen peroxide and other ionization elements to pass through to the backside of the ridges 165. This air exchange through the holes 153 also helps to dissipate heat generated by the bulb 160. In some embodiments, the ends of the hollow sections 159 and the holes 153 may be closed off such that essentially all airflow is passed through the troughs 166 that are hit directly with the UV light. However, it should be noted that even airflow not in contact with the UV light at all can still be sufficiently cleansed by the device because the ions and charged particles created travel through the air medium and are chemically attracted to organic compounds that within the air. Once attached to these compounds, the oxidation process takes place leaving them inert.

FIGS. 9 and 9A show an alternative design for a core assembly. Core assembly 250 may be formed as a one-piece design and need not have a separate sleeve. Here, the core assembly has an interior surface featuring a number of oscillating troughs 251 and teeth 252 that are again designed to maximize catalyst-coated surface area exposed to the UV light source and to bring portions of the surface in closer proximity to said source. However, if a one-piece design, it is more difficult to consistently coat the inner surface of core assembly 250. A dipping process may be used, but then catalyst is largely wasted on the exterior surface. In some embodiments, core assembly 250 may have a parting line down one side such that it may be unrolled so that it can be sprayed with catalyst only along the inside surface, then rolled up and inserted into a rigid sleeve similar to sleeve 152 of FIG. 8.

FIG. 10 illustrates a closeup view of the filter assembly 170 shown in FIG. 2. The filter assembly includes a filter tray 171 into which a filter medium 172 slides. The filter medium is a replaceable, membrane through which air can easily pass, but which collects and filters out larger particles such as dust, pollens, and fibrous materials within the air. The filter assembly has an extraction handle 173 that can be gripped by a user to remove it from the filter tray housing 175 (see FIG. 2) for replacement or cleaning. In the illustrated embodiment, the filter assembly 170 is positioned below the core assembly 150 and fan assembly 140. However, so long as it is positioned within the air pathway between the air inlets 191 and air outlets 192, the filter assembly can serve its purpose.

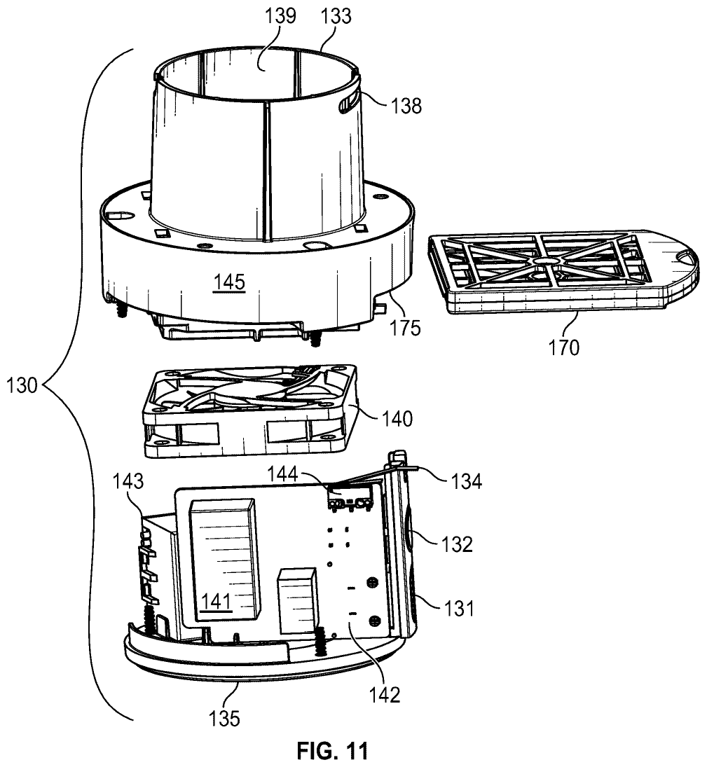

FIG. 11 illustrates certain components of the base structure 130 according to the illustrated embodiment. Here, filter assembly 170 has been extracted from its housing 175, and the fan assembly 140 has been dropped down below the fan housing 145 where it is positioned when fully assembled. The top portion of the base structure 130 forms the core assembly housing 139 for receiving the core assembly 150 (not shown). Below the fan housing is the portion of the base structure 130 that packaged inside of the perimeter extensions 136 and protrusions 147 shown in FIG. 3. These components include a circuit board 142 that provides the electrical connections between the power receptacle 131, power button 132, light switch 144 and the fan motor 141. As shown, light switch 144 is connected to the safety catch 134, which, as described above, operates to cut power to the UV bulb 160 when the outer sleeve 120 is removed. The illustrated embodiment also includes a backup battery pack 143 for operation when not connected to a wall socket.

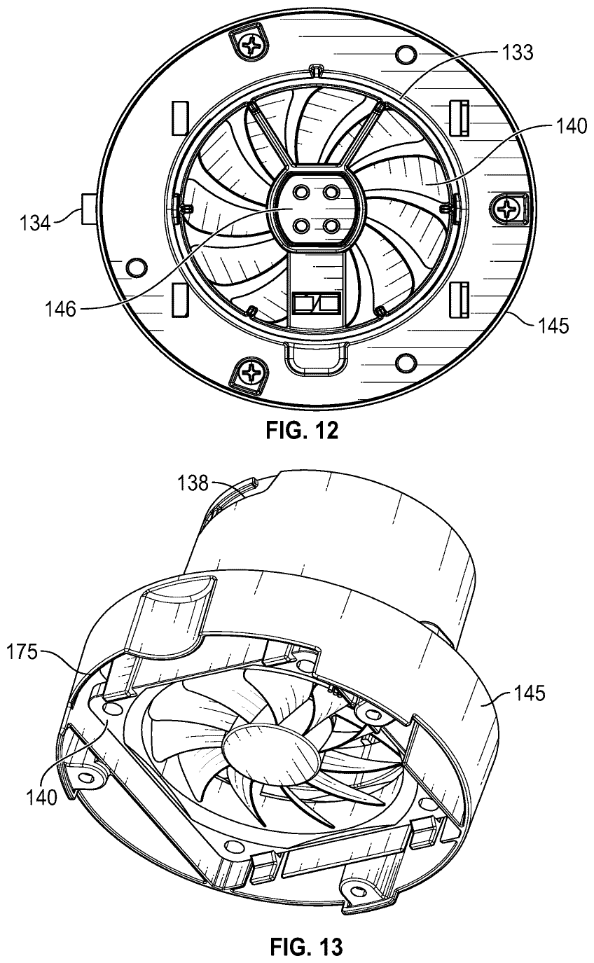

FIG. 12 shows a top view of the base structure 130 in isolation with the core assembly 150 and bulb 160 removed. The core assembly housing 139 lies within the upper rim 133 and is open at the bottom through which the fan assembly 140 is visible within fan housing 145. At the center is light receptacle 146. When installed, the pins 163 of bulb 160 fit into the light receptacle 146 to connect the bulb to a power source and hold it in position. FIG. 13 rotates this same isolated portion of the base structure 130 so as to view the bottom. This shows the fan assembly 140 installed up in the fan housing 145. The filter assembly 170 has been removed, but would slide in to filter tray housing 175 beneath the fan in the illustrated embodiment.

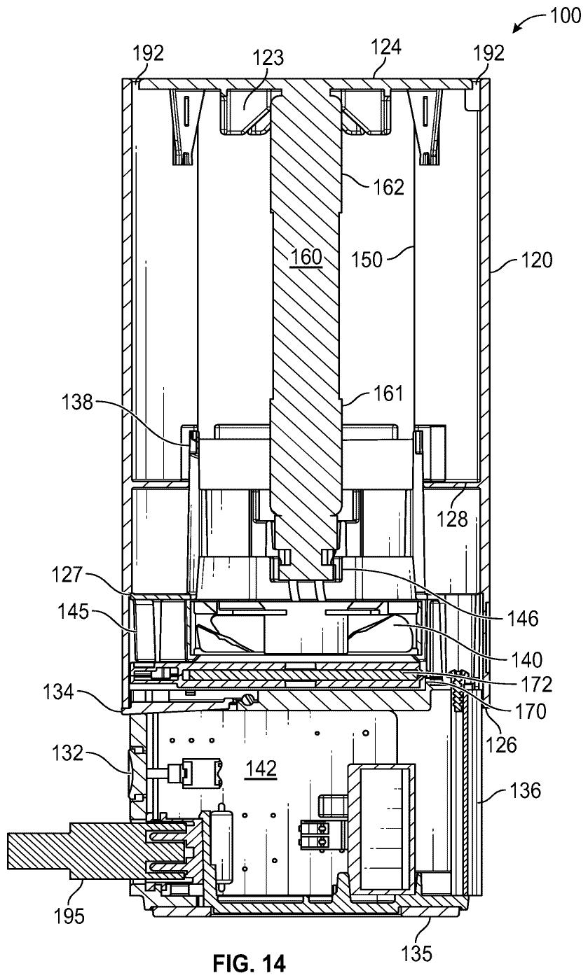

FIG. 14 provides a section view down the center of the air purification device 100. This provides a view of all of the internal components in their assembled position. The air purification unit 100 sits on its bottom surface 135 and extends upward through the base structure that includes the circuit board 142 and other electrical components powered through electrical cable 195. Air passes in under the lower perimeter 126 of the outer cover 120, up through the filter medium 172, and through the fan assembly 140. It then continues up along the outside of bulb 160 and through the core assembly 150, eventually passing along the bottom of top surface 124 and out through air outlets 192.

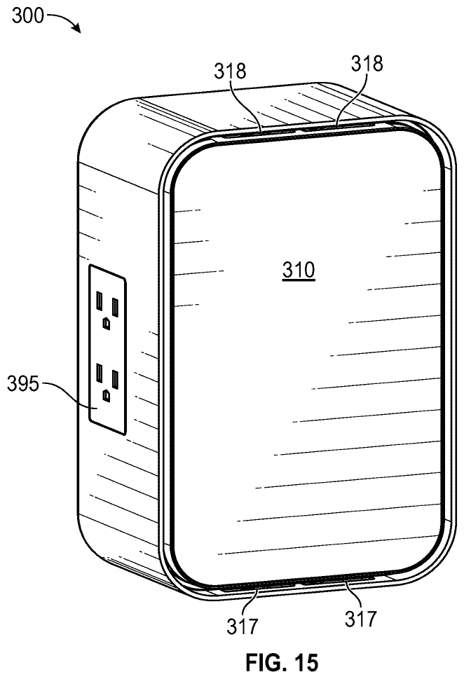

FIGS. 15-19 show a different embodiment of an air purification unit that incorporates various concepts of the present invention. While the embodiment previously discussed is designed to be portable, the embodiment of FIGS. 15-19, referred to as a “wall unit,” is generally intended to be mounted to a wall, ceiling or other fixed construction. Though having a completely different external shape and appearance, as will be seen, the “wall unit” shares many internal components with the mobile unit previously discussed.

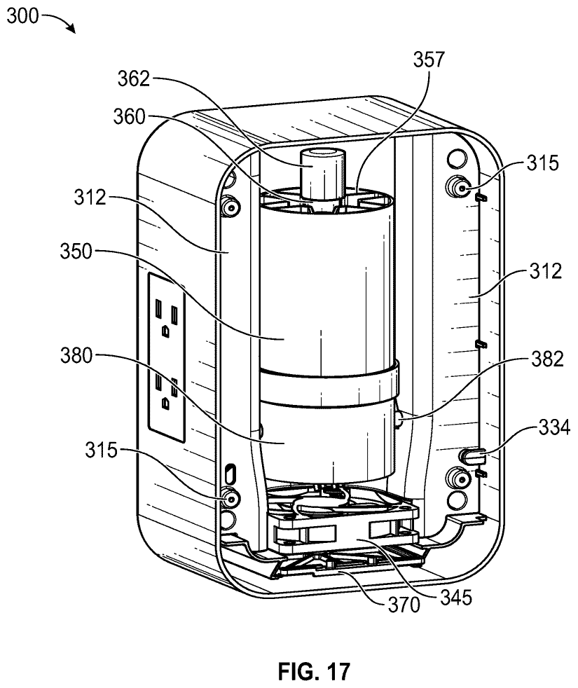

As shown in FIG. 15, the “wall” air purifier unit 300 is of essentially rectangular construction, having a back panel 320, side walls providing for depth, and a face plate 310 on front. The face plate 310, which is substantially opaque and removable just like outer cover 120 of the previously discussed embodiment, is configured to fit inside the perimeter of the side walls of the unit 300. In the illustrated embodiment, the face plate 310 is held in place by magnets 315 (see FIG. 17) positioned on forward panels 312 of the unit 300. However, a variety of common mechanical fasteners could be used to attach and retain the fac plate, such as screws, clips, etc. At the bottom of the face plate 310 are a couple of air intake ports 317, and at the top of the face plate are a couple of air outlet ports 318. These ports could be of various sizes, shapes or quantities, the important aspect being that they allow for a generally uni-directional air flow through the air purifier unit 300. In order to help keep the air within the pathway from input to output, the face plate 310 may be equipped with foam inserts that seat up against the forward panels 312 (shown in FIG. 17) when the face plate is installed.

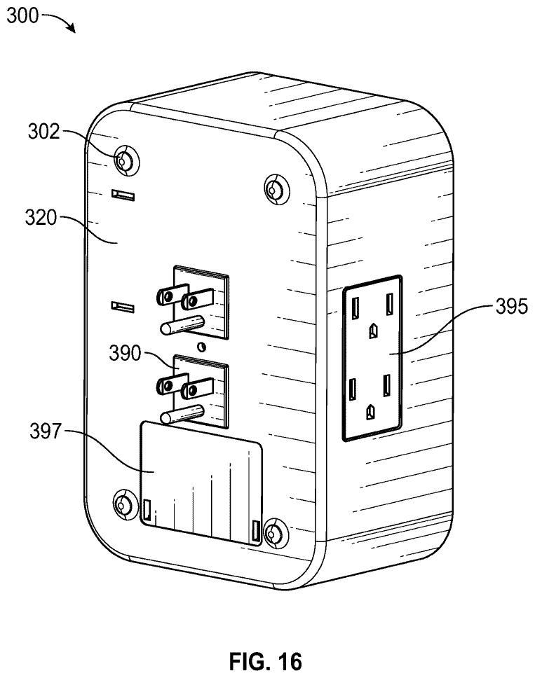

FIG. 16 illustrates a rear angle view of the air purifier unit, showing the back panel 320. The back panel is configured for mounting against a flat structural surface such as a wall or ceiling using one of four attachment holes 302. These holes may be accessed from the inside of the air purification unit 300, and common screws may be used to fix the air purification unit in place. In the illustrated embodiment, the unit is mounted over the top of a common electrical outlet, and the unit is equipped with one or two wall plugs 390 that fit into the outlet. In some cases, for smaller embodiments such as that shown, the unit 300 may remain in place simply through retention of the plugs 390 in the socket (not shown) such that screws and holes 302 are not used.

The purpose of having two plugs 390 is to allow access to electricity for other devices despite the outlet being fully covered by the device. That is, one of the plugs 390 is used to receive power to operate the air purifier unit 300, while the other plug 390 connects power to the pass through socket 395 on the side of the unit 300. This pass through socket 395 is then available for use by other devices. Air purifier unit 300 may also be equipped with batteries to power the device during times of electrical failure. As shown a battery compartment cover 397 conceals where the batteries could be installed.

FIG. 17 shows certain internal components of the air purification unit 300, also referred to herein as the “wall unit.” As will be seen, many of these internal components are similar to or the same as internal components of the previously disclosed purification unit in FIGS. 1-14, such that the parts may even be interchangeable. At the bottom near where air enters the intakes 317 is an air filter assembly 370. This air filter assembly may have the same basic components and structure as previously disclosed air filter assembly 170, so it will not be revisited here. Like air filter assembly 170, air filter assembly 370 is removable and may be replaced or cleaned. Just above air filter assembly 370 is fan housing 345. This fan housing is similar to, and may be interchangeable with, fan housing 145 of the previously disclosed embodiment. As shown, it is positioned between the forward panels 312 within the unit 300 so that air is generally directed up through the fan assembly 340 toward the core assembly 350 above.

Once through the fan, air is directed up through a harness 380, which has a perforated bottom and is used to mount the core assembly 350. Core assembly 350 can be interchangeable with core assembly 150 of the mobile unit 100. That is, it features a sleeve 352 that contains an ionization cell 357 having a quad-metallic catalyst coating on an inner surface, troughs and ridges, and seams that allow it to fold around a UV light source. While the size and dimensions of these components may vary, the same description provided in association with FIGS. 6-8 above applies to the core assembly 350 of the wall unit 300. In addition, the bulb 360 is of the same variety as bulb 160. That is, it has opaque top and bottom caps (362, 361) and a central glass or plastic piece that is translucent, allowing the UV Light to pass through and interact with the catalyst of the ionization cell 357 wrapped around it. At bottom of the light 360 are connector pins 363 that plug into a light receptacle 346, with wiring leading to the electrical source.

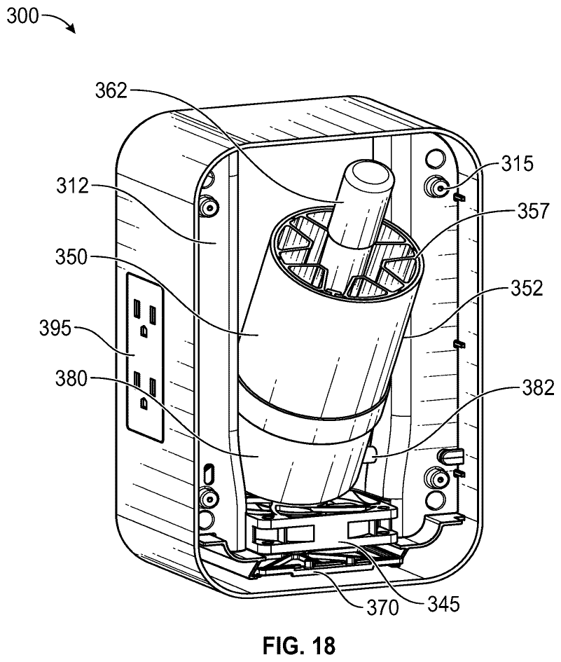

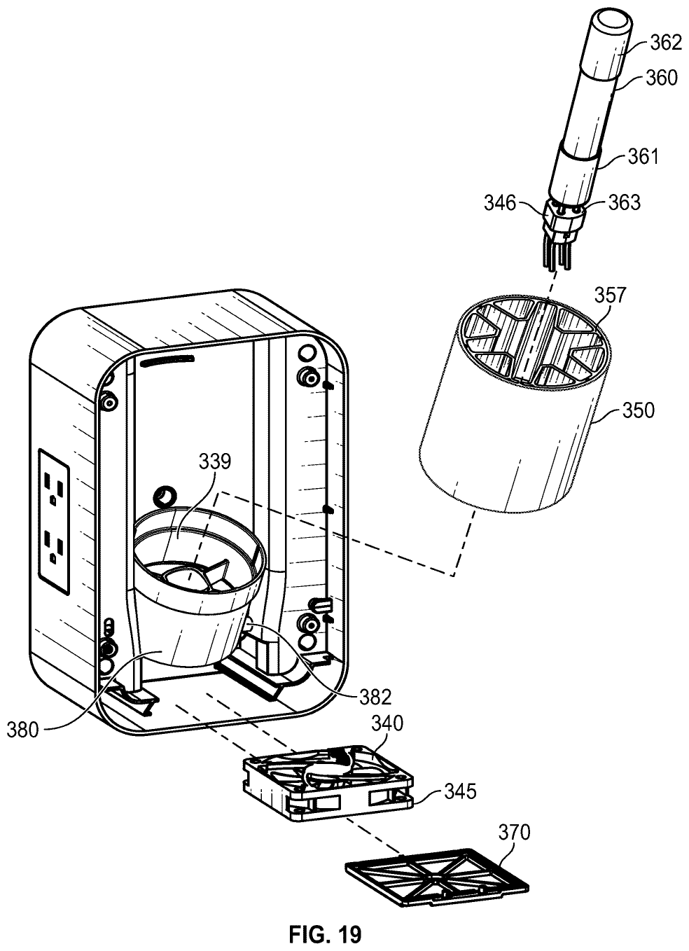

A key difference between the mobile unit and the wall unit is the presence and function of harness 380. With the mobile unit, there is no upper blockage when the outer cover 120 is removed. However, even when face plate 310 is removed, the bulb 360 and ionization cell 357 may not be removed for servicing or replacement because the top panel of the unit 300 is still in the way. To overcome this, wall unit 300 features harness 380, which takes the place of the fixed core assembly housing 139 of the mobile unit. Instead, as shown in FIG. 19, the harness 380 provides a core assembly housing 339, and the entire harness 380 may be rotate outward of the wall unit structure along axle 382 such that the core assembly 350 and bulb 360 may be removed. FIG. 18 shows the same view as FIG. 17 with the face plate 310 removed, but the harness 380 in FIG. 18 has been rotated out to illustrate this. FIG. 19 shows the view of FIG. 18, but with the core assembly 350 and the bulb 360 exploded out to reveal the hollow bottom of the harness 380 through which air passes through from the fan assembly 340 below.

Though not shown, wall air purifier unit 300 is equipped with a circuit board, motor, and electrical switches connected to the fan assembly 340 and the UV bulb 360 just as in the previously disclosed mobile unit 100. However, it will be understood that different motor and circuit board geometries could be designed or selected to fit within the different physical space provided. Also, the wall unit 300 can utilize a pressure switch 334 that operates like the safety catch 134 of the mobile unit 100. That is, when the face plate 310 is removed from the unit, the pressure switch 334 is released, which opens a switch and shuts off the flow of electricity to the bulb 360 so as to prevent external exposure to ultraviolet light from the bulb 360.

Though a reasonably compact wall unit is disclosed, the size of the wall unit is easily scaled. For example, while still using the same size bulb 360, core assembly 350 and harness 380, a number of these components could be positioned side by side, each with individual fan assemblies 340 and air filters 370 underneath them, separated by structure similar to forward panels 312 to create independent air flow paths through each modular unit. A number of these could be positioned side by side within a longer panel wall unit to effectively cleans a much larger open area.

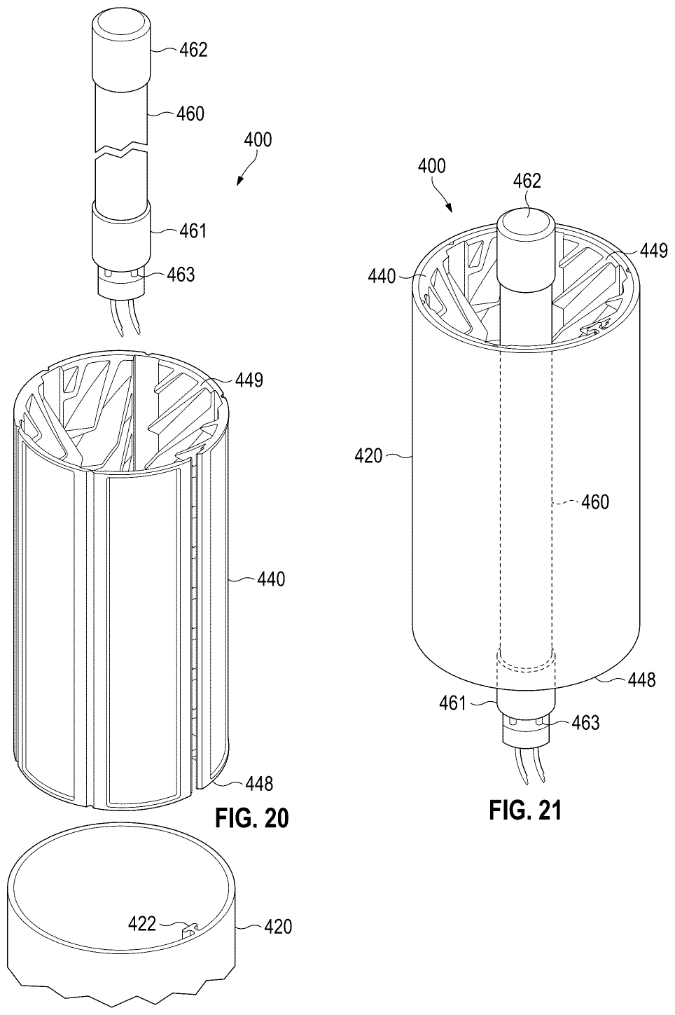

FIG. 20 shows an exploded view of a core assembly 400 for an air purification unit such as the one illustrated in FIGS. 1-3, according to an example configuration. As shown, core assembly 400 includes a sleeve 420 and an ionization cell 440. The ionization cell 440 is configured to fit within the sleeve 420, and the sleeve 420 further includes a rib 422 to hold the ionization cell 440 in place and prevent it from rotating, as described in further detail below. Additionally, the ionization cell 440 has a bottom end 448 and a top end 449. The bottom end 448 can be understood as being nearest an inlet for air, whereas the top end 449 can be understood as being nearest an outlet. Just as described with regard to alternative configurations throughout the disclosure, the core assembly 400 is structured to receive a bulb 460. Bulb 460 is a source of ultraviolet light in the example configuration shown in FIG. 20, having the same or similar properties of bulb 160 described above with regard to FIG. 2. Additionally, bulb 460 has a bottom cap 461 and a top cap 462 that are generally opaque. The portion of bulb 460 extending between the bottom cap 461 and the top cap 462 is translucent, such that light only emanates from bulb 460 between the bottom cap 461 and the top cap 462. Bulb 460 also includes electrical connection 463 for powering the bulb 460.

As mentioned with regard to other configurations of the disclosure, bulb 460 fits inside core assembly 400 along a central axis of core assembly 400, as shown in FIG. 21, and ionization cell 440 has catalyst disposed on a surface facing bulb 460. In operation, both core assembly 400 and bulb 460 fit into a base structure of an air purification unit. Although such a base structure and air purification unit are not illustrated in FIGS. 20-21, it should be noted that core assembly 400 as illustrated in FIGS. 20-21 can be implemented with an air purification unit in the same or similar manner as configurations of the core assembly described with regard to FIG. 2. Additionally, in configurations, sleeve 420 is substantially opaque. For purposes of this disclosure, “substantially opaque” means largely or essentially blocking the passage of radiant energy or light, without requiring perfect opacity. In this way, sleeve 420 is configured to block view of the ultraviolet light source from outside the air purification unit implementing core assembly 400.

Accordingly, in implementation, core assembly 400 of FIGS. 20-21 operates similarly to configurations of core assembly described throughout the disclosure. Namely, core assembly 400 can be implemented with an air purification unit such that, when the air purification unit is powered on, air is drawn from the ambient environment into the air purification at an end nearest the bottom end 448 of the ionization cell 440. The air enters the core assembly 400 and flows toward the top end 449 in a spiral path directed by structural features of the ionization cell 440, described in further detail below. As the air flows through core assembly 400, the catalyst disposed on a surface of the ionization cell 440 facing bulb 460 interacts with the high intensity ultraviolet light of bulb 460 to produce hydrogen peroxide ions and other oxidizing agents. These ions break down further into water and air and reduce air pollutants and neutralize viruses in the process. Once the flow of air reaches an of the core assembly 400 nearest the top cap 462 of bulb 460, the air exits the core assembly 400 to be released back into the ambient environment purified of such pollutants and viruses.

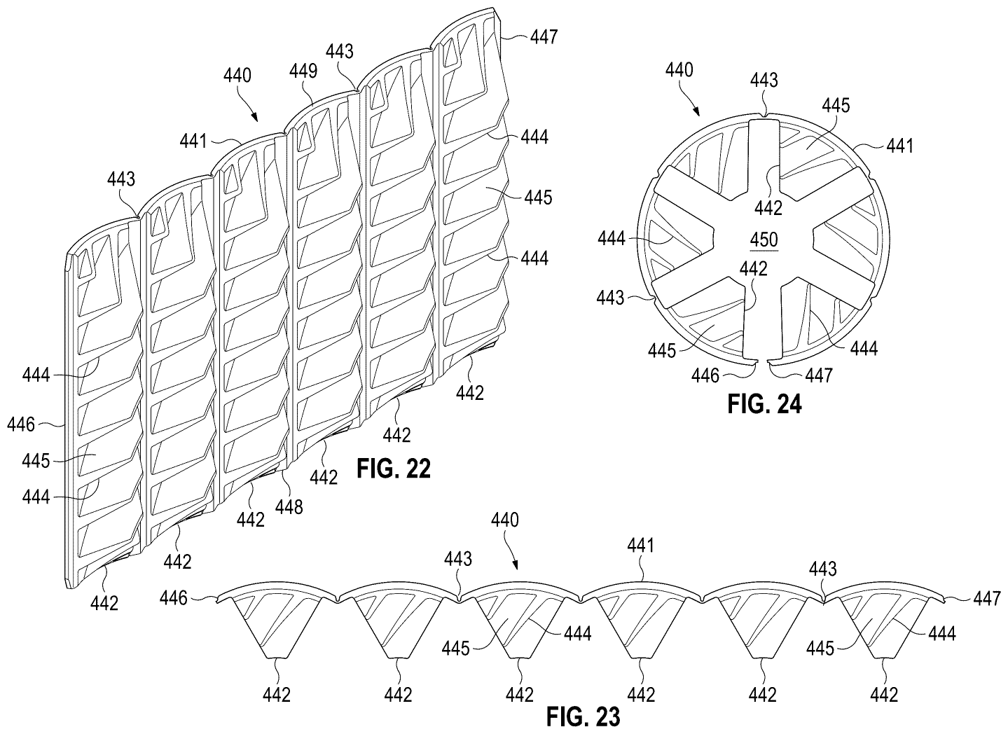

As mentioned, core assembly 400 has an ionization cell 440 with geometry structured to direct the flow of air in a spiral path. FIG. 22 shows a perspective view of ionization cell 440 in an unfolded position, illustrating further details of this geometry. In the unfolded position, ionization cell 440 is substantially flat. For the purposes of this disclosure, “substantially flat” means largely or essentially flat, without requiring perfect flatness. Similar to configurations described above, ionization cell 440 has a plurality of backing elements 441, supporting a plurality of cell components 443. Each of backing elements 441 forms an arc connecting foldable seams 443 on either side a cell component. In this way ionization cell 440 can be understood as a plurality of cell components 443 supported by backing elements 441 and joined by foldable seams 443. Although illustrated as flat in FIG. 22, ionization cell 440 is structured to fold at each of the foldable seams 443 to allow ionization cell 440 to fit within the overall core assembly, as shown in FIGS. 20-21. Specifically, as described in further detail below, a first edge 446 of ionization cell 440 can be brought toward a second edge 447, causing each of foldable seams 443 to fold.

Referring once again to FIG. 22, a plurality of fins 444 are disposed along the length of each of cell components 442 in a repeating pattern, from the bottom end 448 of the ionization cell 440 to the top end 449. As shown, fins 444 each extend from cell components 442 at an angle. Specifically, fins 444 extend from cell components 442 such that an angle may be measured from the bottom end 448 of the ionization cell 440 to the line at which each of fins 444 connects to cell components 442. In configurations, the angle at which fins 444 extend from cell components 442 ranges from 5° to 45°. Preferably, in configurations, the angle at which fins 444 extend from cell components 442 ranges from 30° to 45°. Additionally, in configurations, fins 444 are shaped to be triangular, with the longest leg of the triangle forming the line at which each of fins 444 connects to the cell components 442. In still other configurations, fins 444 are shaped to be trapezoidal, with the fins connecting to cell components 442 along a base of the trapezoid.

As mentioned, fins 444 are disposed along the length of each of cell components 442 in a repeating pattern. In this way, fin channels 445 are formed between each of the fins 444 disposed on the same cell component. Fin channels 445 are more clearly illustrated in FIG. 23, which shows a top view of ionization cell 440 in an unfolded position. In configurations, the distance between each of fins 444 disposed on the same cell component-and, accordingly, the width of the fin channels 445 formed between them-ranges from 2 mm to 10 mm. Preferably, the fins 444 are disposed on cell components 442 such that they are as close to each other as possible, subject to constraints of the manufacturing tools used to form the fins.

Similar to configurations of the disclosure discussed above, ionization cell 440 is coated with a catalytic material, at least on a surface on which fins 444 are disposed. Because ionization cell 440 is foldable and unfoldable at foldable seams 443, the catalytic material can be applied to the desired surface during the manufacturing process without wasting material on a surface of ionization cell 440 opposite the fins 444-namely, the surface comprising backing elements 441. For instance, the catalytic coating may be applied to ionization cell 440 with spray process, using less material than would be used if the entire ionization cell 440 were dipped in the catalytic material. This, in turn, reduces labor and costs associated with coating the ionization cell 440.

FIG. 24 shows a top view of ionization cell 440 in a folded position, according to configurations. As shown, first edge 446 of the ionization cell 440 has been brought toward the second edge 447, causing each of the foldable seams 443 to fold. As viewed from the top, as illustrated in FIG. 24, the arc-shaped backing elements 441 form a shape that is substantially circular. With reference back to FIGS. 20-21, the ionization cell 440 is thus shaped to be substantially cylindrical in the folded position. For the purposes of this disclosure, “substantially circular” means largely or essentially resembling a circle, without requiring perfect circularity, and “substantially cylindrical” means largely essentially resembling a cylinder, without requiring perfect cylindricality.

As shown in FIG. 24, fins 444 extend from their corresponding cell components 442 toward a central axis of the folded ionization cell 440, but fins 444 of one cell component do not contact fins 444 of a neighboring cell component. In this way, fins 444 extend toward the central axis but leave open a central channel 450 along the central axis. Central channel 450 is thus structured to receive an ultraviolet bulb when the folded ionization cell 440 is implemented with a core assembly, in configurations. Additionally, when in the folded position illustrated in FIG. 24, the fins 444 are oriented such that the fins 444 of one cell component direct a flow of air toward a neighboring cell component, and so on. This path of air flow is dependent on the specific orientation of the fins 444 and is described in further detail below.

To enable ionization cell 440 to fold into the position shown in FIG. 24, ionization cell 440 may be made of a semi-flexible material such as polycarbonate, polyamide, or polypropylene. Other plastics, paper pulp, or other natural and compostable materials could also be used. Material selection for ionization cell 440 ideally allows a level of flexibility at foldable seams 443 to accommodate the folded position, but sufficient firmness for the ionization cell 440 to hold its shape as it is inserted into or extracted from the core assembly. Additionally, material must be selected to withstand heat and radiation from the ultraviolet light source over long periods of time.

Due to the geometry and dimensions of fins 444, ionization cell 440 has a large surface area on which the catalytic coating is applied. Accordingly, when air is drawn into core assembly 400 having an ionization cell 440, air flowing past fins 444 and through fin channels 445 remains exposed to the catalytic coating for most of its path. For instance, without such fins 444, a portion of air may travel close to bulb 460 but an undesirable distance from any surface coated with the catalyst. Having fin channels 445 to direct the flow of air ensures a large majority of the air flowing through the core assembly is exposed to a surface coated with catalyst.

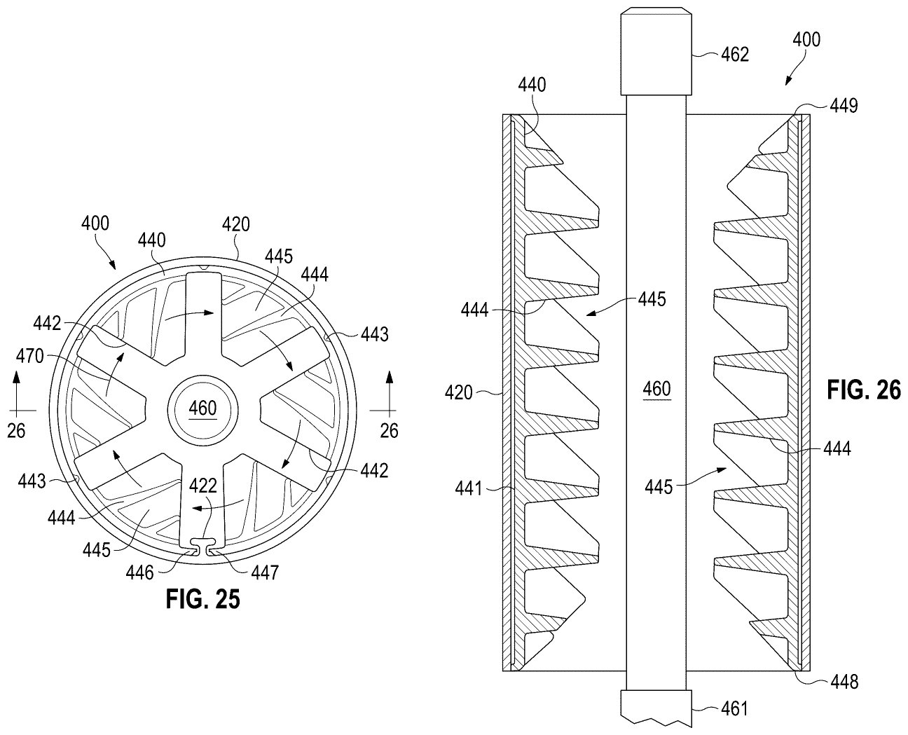

The geometry and dimensions of fins 444 and fin channels 445 also direct the flow of air in a spiral path, increasing the exposure time of the air to the catalytic coating and the ultraviolet light relative to a linear path. This spiral path is illustrated in more detail in FIG. 25, which shows a top view of core assembly 400 having ionization cell 440 and bulb 460 installed in a sleeve 420. As shown, ionization cell 440 is installed within the sleeve 420 by bringing first edge 446 and second edge 447 toward each other to fold the foldable seams 443 and make ionization cell 440 substantially cylindrical. When first edge 446 and second edge 447 are brought in close proximity, these edges fit under opposing sides of rib 422 as ionization cell 440 is slid into the sleeve 420. Ionization cell 440 is slid into sleeve 420 with backing elements 441 abutting an inner surface of the sleeve 420 and with fins 444 extending toward bulb 460. Bulb 460, as shown, is installed along the central axis of the core assembly 400, previously shown as the central channel 450 of FIG. 24. Thus, when the ionization cell 440 is in the folded position and installed in the core assembly 400, the ionization cell 440 substantially surrounds the bulb 460. For purposes of this disclosure, to “substantially surround” means to largely or essentially extend around, without requiring perfect encircling.

With fins 444 extending at angles away from each of the cell components 442, air is directed in a spiral path through the core assembly 400, in configurations. This spiral path is represented with arrow 470, for clarity. More specifically, air drawn into the core assembly 400 implementing ionization cell 440 tends to flow through fin channels 445. Because fin channels 445 are shaped-through the geometry of fins 444, described above—to angle toward the top end 449 of ionization cell 440, air tends to flow through fins channels 445 with the angles toward the top end 449. And, because fin channels 445 of one cell component terminate at the next highest fin channels 445 of a neighboring cell component, air continues to flow at the angle directed by fin channels 445 as it moves from the bottom end 448 to the top end 449. The repeating pattern of fins 444 and fin channels 445 disposed on the ionization cell 440 thus causes air to follow a spiral path as it flows from the bottom end 448 to the top end 449.

FIG. 26 shows a cross-sectional view of core assembly 400, according to the cross-section identified in FIG. 25. In particular, FIG. 26 shows further detail of the geometry and orientation of fins 444 and fin channels 445 within the overall core assembly 400. Although just two cell components are illustrated in FIG. 26, with one on either side of bulb 460, it should be understood that additional cell components having fins 444 and fin channels 445 are disposed about the core assembly 400. Any cell components, fins 444, and fin channels 445 hidden from view in FIG. 26 are for clarity of illustration and are not intended to be limiting.

With reference to FIG. 26, air can be drawn into an air purification unit implementing core assembly 400. Specifically, air is drawn into the core assembly 400 nearest the bottom end 448 of ionization cell. Upon entry, fins 444 nearest the bottom end 448 cause the air to follow the path formed by fin channels 445, angling the flow of air relative to the bottom end 448. Once air flows through and past fin channels 445 nearest the bottom end 448, air is further guided by fins 444 and fin channels 445 immediately above—i.e., the next closest fins 444 and fin channels 445 relative to the bottom end 448. Air continues to flow in this pattern as it flows farther from the bottom end 448 and closer to the top end 449, ultimately creating a spiral path. Once the flow of air reaches the fins 444 and fin channels 445 nearest the top end 449, it exits the core assembly 440 to be dispersed back into the ambient environment.

Additionally, as shown in FIG. 26, bulb 460 extends along the entire length of core assembly 400 when installed. For instance, bottom cap 461 and top cap 462 of bulb 460, which are opaque and do not emanate ultraviolet light, are located outside the bounds of the core assembly 400 when installed. Consequently, the portion of bulb 460 within the bounds of core assembly 400 emanates ultraviolet light, and air flowing through core assembly 400 is thus exposed to ultraviolet light for its entire travel path.

Moreover, the spiral path of the air flowing from the bottom end 448 to the top end 449 enables air to be brought as close as possible to bulb 460 without being brought too far from surfaces having the catalytic coating. Closer proximity to bulb 460 strengthens the photocatalysis process. And, as mentioned, fins 444 increase the surface area of ionization cell 440 and provide a greater amount of surface having the catalytic coating than if fins 444 were not present. Accordingly, the travel path guided by fins 444 maximizes the influence of both proximity to ultraviolet light and proximity to catalytic material.

The spiral path of the air, as mentioned, also increases the amount of time air spends in the core assembly 400 compared to if the air followed a linear path from the bottom end 448 to the top end 449. This increased travel duration causes air to experience greater exposure duration to the ultraviolet light and catalytic coating, further maximizing the effects of the photocatalysis process within the core assembly 400. More specifically, increasing the exposure time increases the probability of a stabilized reaction to generate more hydrogen peroxide ions, thus increasing the purification potential of the core assembly 400.

As shown in FIG. 26, fins 444 extend a radial distance toward bulb 460, but a gap remains between bulb 460 and the fins 444. Preferably, in configurations, this gap between bulb 460 and fins 444 ranges from 1 to 3 mm. Ideally, this gap is kept as small as possible to maximize exposure to both the ultraviolet light of bulb 460 and the catalytic coating on fins 444, and to maximize the amount of air flowing in the spiral path directed by fins 444. Put differently, a large gap between bulb 460 and fins 444 could allow more air to travel in a linear path along bulb 460, decreasing travel time and decreasing exposure to the catalytic coating.

Those skilled in the mechanical arts will appreciate that various changes may be made and equivalents may be substituted without departing from the scope of systems and methods disclosed in this application. For example, the air purification units can take other exterior physical shapes without departing from the internal components of the present invention discussed herein. As mentioned, the unit may be scaled to handle larger or smaller volumes of air. The order of the fan, air filter and light bult/ionization cell combination can be in any order so long as they are positioned between an air inlet and an air outlet, and a generally closed channel is provided for air to pass from inlet to outlet. Various cell geometries could also be used without departing from the spirit of the invention, the general effort being to maximize the exposed surface area while minimizing distance to the ultraviolet light source. The catalyst coating could be varied in several respects such as parts per million and ratio of metals so long as it still produces the charged ions to inspire oxidation. The selection of materials for the physical components of the device may be altered based on cost, appearance, and durability requirements. Additionally, although configurations of the disclosed technology are described as air purification units, it should be noted that the disclosed devices and methods are not limited to purifying air alone. For instance, the disclosed devices and methods for drawing in and treating air act to purify spaces and surfaces, by extension of the treatment of air. Thus, it is intended that the novel techniques of the present invention not be limited to the particular embodiments explicitly disclosed, but that they include all techniques falling within the scope of the appended claims.

### EXAMPLES

Illustrative examples of the disclosed technologies are provided below. A particular configuration of the technologies may include one or more, and any combination of, the examples described below.

Example 1 includes an air purification unit comprising: an air inlet; an air outlet separated a distance from the air inlet; an ultraviolet light source positioned along a length of the distance; and an ionization cell having an inner surface coated with a catalyst, the inner surface having a plurality of fins disposed in a repeating pattern and extending from the inner surface of the ionization cell at an angle relative to an end of the ionization cell nearest the air inlet.

Example 2 includes the air purification unit of Example 1, in which the plurality of fins is structured to form a plurality of fin channels that cause air to travel in a spiral path from the air inlet to the air outlet.

Example 3 includes the air purification unit of any of Examples 1-2, in which the ionization cell comprises a plurality of cell components, each of the cell components having a portion of the plurality of fins disposed in a linear pattern along a direction parallel to the length of the distance between the air inlet and the air outlet.

Example 4 includes the air purification unit of any of Examples 1-3, in which the plurality of fins extend from the inner surface of the ionization cell at an angle ranging from 5 degrees to 45 degrees relative to the end of the ionization cell nearest the air inlet.

Example 5 includes the air purification unit of any of Examples 1-4, further comprising a fan to move air from the air inlet, along the length, and out the air outlet.

Example 6 includes the air purification unit of Example 5, further comprising: a base structure for housing the fan; and a substantially opaque removable cover that, when in an installed position, blocks view of the ultraviolet light source from outside the air purification unit.

Example 7 includes the air purification unit of Example 6, further comprising a switch controlling the flow of electricity to the ultraviolet light source, wherein the switch only allows the flow of electricity to the ultraviolet light source when the substantially opaque removable cover is in the installed position.

Example 8 includes the air purification unit of any of Examples 6-7, wherein the substantially opaque removable cover comprises an open bottom end that allows air intake to the fan and a top end with at least one opening to allow air to escape the air purification unit after passing through the ionization cell.

Example 9 includes the air purification unit of any of Examples 1-8, wherein ultraviolet light produced by the ultraviolet light source reacts with the catalyst to produce ions that oxidize microbes in air passing through the ionization cell.

Example 10 includes an air purification unit, comprising: an air inlet; an air outlet separated a distance from the air inlet; an ultraviolet light source positioned along a length of the distance; and a removable ionization cell having a plurality of fins disposed on an inner surface coated with a catalyst, the ionization cell further having an unfolded position wherein the ionization cell is substantially flat and a folded position wherein the ionization cell is configured to substantially surround the ultraviolet light source.

Example 11 includes the air purification unit of Example 10, in which the plurality of fins is structured to form a plurality of fin channels that cause air to travel in a spiral path from the air inlet to the air outlet.

Example 12 includes the air purification unit of any of Examples 10-11, in which the removable ionization cell comprises a plurality of cell components, each of the cell components having a portion of the plurality of fins disposed in a linear pattern along a direction parallel to the length of the distance between the air inlet and the air outlet.

Example 13 includes the air purification unit of any of Examples 10-12, further comprising a fan to move air from the air inlet, along the length, and out the air outlet.

Example 14 includes the air purification unit of Example 13, further comprising: a base structure for housing the fan; and a substantially opaque removable cover that, when in an installed position, blocks view of the ultraviolet light source from outside the air purification unit.

Example 15 includes the air purification unit of Example 14, further comprising a switch controlling the flow of electricity to the ultraviolet light source, wherein the switch only allows the flow of electricity to the ultraviolet light source when the substantially opaque removable cover is in the installed position.

Example 16 includes the air purification unit of any of Examples 10-15, wherein ultraviolet light produced by the ultraviolet light source reacts with the catalyst to produce ions that oxidize microbes in air passing through the ionization cell.

Example 17 includes an air purification unit, comprising: a sleeve having an air inlet end and an air outlet end, the sleeve structured to be substantially cylindrical; an ultraviolet light source positioned along a central axis of the sleeve; and a removable ionization cell having a plurality of fins disposed on an inner surface coated with a catalyst, the plurality of fins structured to form a plurality of fin channels that cause air to travel in a spiral path from the air inlet end to the air outlet end, and the ionization cell further having an unfolded position wherein the ionization cell is substantially flat and a folded position wherein the ionization cell is configured to fit within the sleeve and substantially surround the ultraviolet light source.

Example 18 includes the air purification unit of Example 17, in which the plurality of fins extend from the inner surface at an angle relative to an end of the ionization cell nearest the air inlet end when the removable ionization cell is installed within the sleeve.

Example 19 includes the air purification unit of any of Examples 17-18, in which the sleeve is substantially opaque and blocks view of the ultraviolet light source from outside the air purification unit.

Example 20 includes the air purification unit of any of Examples 17-19, in which ultraviolet light produced by the ultraviolet light source reacts with the catalyst to produce ions that oxidize microbes in air passing through the ionization cell.

Aspects may operate on a particularly created hardware, on firmware, digital signal processors, or on a specially programmed general purpose computer including a processor operating according to programmed instructions. The terms “controller” or “processor” as used herein are intended to include microprocessors, microcomputers, ASICs, and dedicated hardware controllers. One or more aspects may be embodied in computer-usable data and computer-executable instructions, such as in one or more program modules, executed by one or more computers (including monitoring modules), or other devices. Generally, program modules include routines, programs, objects, components, data structures, etc. that perform particular tasks or implement particular abstract data types when executed by a processor in a computer or other device. The computer executable instructions may be stored on a non-transitory computer readable medium such as a hard disk, optical disk, removable storage media, solid state memory, RAM, etc. As will be appreciated by one of skill in the art, the functionality of the program modules may be combined or distributed as desired in various configurations. In addition, the functionality may be embodied in whole or in part in firmware or hardware equivalents such as integrated circuits, field programmable gate arrays (FPGA), and the like. Particular data structures may be used to more effectively implement one or more aspects of the disclosed systems and methods, and such data structures are contemplated within the scope of computer executable instructions and computer-usable data described herein.

The previously described versions of the disclosed subject matter have many advantages that were either described or would be apparent to a person of ordinary skill. Even so, all of these advantages or features are not required in all versions of the disclosed apparatus, systems, or methods.

Additionally, this written description makes reference to particular features. It is to be understood that the disclosure in this specification includes all possible combinations of those particular features. For example, where a particular feature is disclosed in the context of a particular example configuration, that feature can also be used, to the extent possible, in the context of other example configurations.

Also, when reference is made in this application to a method having two or more defined steps or operations, the defined steps or operations can be carried out in any order or simultaneously, unless the context excludes those possibilities.

Furthermore, the term “comprises” and its grammatical equivalents are used in this application to mean that other components, features, steps, processes, operations, etc. are optionally present. For example, an article “comprising” or “which comprises” components A, B, and C can contain only components A, B, and C, or it can contain components A, B, and C along with one or more other components.

Also, directions such as “vertical,” “horizontal,” “right,” and “left” are used for convenience and in reference to the views provided in figures. But the air purification unit may have a number of orientations in actual use. Thus, a feature that is vertical, horizontal, to the right, or to the left in the figures may not have that same orientation or direction in actual use.

Although specific example configurations have been described for purposes of illustration, it will be understood that various modifications may be made without departing from the spirit and scope of the disclosure.

We claim:

1. An air purification unit comprising:
an air inlet;
an air outlet separated a distance from the air inlet;
an ultraviolet light source positioned along a length of the distance; and
an ionization cell having an inner surface coated with a catalyst, the inner surface having a plurality of fins disposed in a repeating pattern and extending from the inner surface of the ionization cell at an angle relative to an end of the ionization cell nearest the air inlet.

2. The air purification unit of claim 1, in which the plurality of fins is structured to form a plurality of fin channels that cause air to travel in a spiral path from the air inlet to the air outlet.

3. The air purification unit of claim 1, in which the ionization cell comprises a plurality of cell components, each of the cell components having a portion of the plurality of fins disposed in a linear pattern along a direction parallel to the length of the distance between the air inlet and the air outlet.

4. The air purification unit of claim 1, in which the plurality of fins extend from the inner surface of the ionization cell at an angle ranging from 5 degrees to 45 degrees relative to the end of the ionization cell nearest the air inlet.

5. The air purification unit of claim 1, further comprising a fan to move air from the air inlet, along the length, and out the air outlet.

6. The air purification unit of claim 5, further comprising:
a base structure for housing the fan; and
a substantially opaque removable cover that, when in an installed position, blocks view of the ultraviolet light source from outside the air purification unit.

7. The air purification unit of claim 6, further comprising a switch controlling the flow of electricity to the ultraviolet light source, wherein the switch only allows the flow of electricity to the ultraviolet light source when the substantially opaque removable cover is in the installed position.

8. The air purification unit of claim 6, wherein the substantially opaque removable cover comprises an open bottom end that allows air intake to the fan and a top end with at least one opening to allow air to escape the air purification unit after passing through the ionization cell.

9. The air purification unit of claim 1, wherein ultraviolet light produced by the ultraviolet light source reacts with the catalyst to produce ions that oxidize microbes in air passing through the ionization cell.

10. An air purification unit, comprising:
an air inlet;
an air outlet separated a distance from the air inlet;
an ultraviolet light source positioned along a length of the distance; and
a removable ionization cell having a plurality of fins disposed on an inner surface coated with a catalyst, the ionization cell further having an unfolded position wherein the ionization cell is substantially flat and a folded position wherein the ionization cell is configured to substantially surround the ultraviolet light source.

11. The air purification unit of claim 10, in which the plurality of fins is structured to form a plurality of fin channels that cause air to travel in a spiral path from the air inlet to the air outlet.

12. The air purification unit of claim 10, in which the removable ionization cell comprises a plurality of cell components, each of the cell components having a portion of the plurality of fins disposed in a linear pattern along a direction parallel to the length of the distance between the air inlet and the air outlet.

13. The air purification unit of claim 10, further comprising a fan to move air from the air inlet, along the length, and out the air outlet.

14. The air purification unit of claim 13, further comprising:
a base structure for housing the fan; and
a substantially opaque removable cover that, when in an installed position, blocks view of the ultraviolet light source from outside the air purification unit.

15. The air purification unit of claim 14, further comprising a switch controlling the flow of electricity to the ultraviolet light source, wherein the switch only allows the flow of electricity to the ultraviolet light source when the substantially opaque removable cover is in the installed position.

16. The air purification unit of claim 10, wherein ultraviolet light produced by the ultraviolet light source reacts with the catalyst to produce ions that oxidize microbes in air passing through the ionization cell.

17. An air purification unit, comprising:
a sleeve having an air inlet end and an air outlet end, the sleeve structured to be substantially cylindrical;
an ultraviolet light source positioned along a central axis of the sleeve; and
a removable ionization cell having a plurality of fins disposed on an inner surface coated with a catalyst, the plurality of fins structured to form a plurality of fin channels that cause air to travel in a spiral path from the air inlet end to the air outlet end, and the ionization cell further having an unfolded position wherein the ionization cell is substantially flat and a folded position wherein the ionization cell is configured to fit within the sleeve and substantially surround the ultraviolet light source.

18. The air purification unit of claim 17, in which the plurality of fins extend from the inner surface at an angle relative to an end of the ionization cell nearest the air inlet end when the removable ionization cell is installed within the sleeve.

19. The air purification unit of claim 17, in which the sleeve is substantially opaque and blocks view of the ultraviolet light source from outside the air purification unit.

20. The air purification unit of claim 17, in which ultraviolet light produced by the ultraviolet light source reacts with the catalyst to produce ions that oxidize microbes in air passing through the ionization cell.
